(19) United States
(12) Patent Application Publication
(10) Pub. No.: US 2010/0082075 A1
(43) Pub. Date: Apr. 1, 2010

(54) DEFIBRILLATOR WITH CPR-VENTILATION ANALYSIS UTILIZING PATIENT PHYSIOLOGICAL DATA

(75) Inventor: James K. Russell, Seattle, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/594,601

(22) PCT Filed: Apr. 4, 2008
(86) PCT No.: PCT/IB08/51282

§ 371 Date: Oct. 5, 2009

Related U.S. Application Data
(60) Provisional application No. 60/911,097, filed on Apr. 11, 2007.

(51) Int. Cl. A61N 1/00; A61B 5/02

(52) U.S. Cl. 607/5; 600/508

(57) ABSTRACT

A defibrillation system and method for treating a heart disorder that includes measuring electrical activity of a patient's heart and processing the measured electrical activity to determine a value descriptive of ventricular fibrillation. The value is compared to a threshold, and according to the comparison of the value to the threshold, an output communicating to the rescuer whether to perform cardio-pulmonary resuscitation with or without ventilation is generated.

This invention relates to systems and methods for providing emergency treatment for heart conditions and, more particularly, to systems and methods for treating ventricular fibrillation.

In sudden cardiac arrest, the patient is stricken with a life threatening interruption to the normal heart rhythm, typically in the form of ventricular fibrillation (VF) or ventricular tachycardia (VT) that is not accompanied by spontaneous circulation (i.e., shockable VT). In VF, the normal rhythmic ventricular contractions are replaced by rapid, irregular twitching that results in ineffective and severely reduced pumping by the heart. If normal rhythm is not restored within a time frame commonly understood to be approximately 8 to 10 minutes, the patient will die. Conversely, the quicker that circulation can be restored (via CPR and defibrillation) after the onset of VF, the better the chances that the patient will survive the event.

The cessation of blood circulation stops delivery of oxygen to all parts of the body. Cerebral hypoxia, or lack of oxygen supply to the brain, causes victims to lose consciousness and to stop normal breathing. Accordingly, treatment of VF may require ventilation, such as by mouth-to-mouth resuscitation. There is often reluctance among lay rescuers to perform mouth-to-mouth resuscitation. Professional rescuers have overcome this reluctance and have barriers or bag-valve masks obviating the problem. However, lay rescuers do not have these means available and may be therefore be reluctant to provide emergency assistance.

In view of the foregoing, there is a need for instructing rescuers in an emergency situation when ventilation should be performed such that unnecessary mouth-to-mouth ventilation is avoided.

One aspect of the invention provides a method for treating a heart disorder that includes measuring electrical activity of a patient's heart and processing the measured electrical activity to determine a value descriptive of ventricular fibrillation. The method further includes comparing the value to a threshold and producing an output according to the comparison of the value to the threshold, the output instructing a rescuer to perform cardio-pulmonary resuscitation with or without ventilation. Another aspect of the invention provides a defibrillator including a pair of electrodes configured to detect electrical signals from a patient, a user interface configured to provide instructions to a rescuer in accordance with interface control signals, and a controller electrically coupled to the electrodes and user interface. The controller is operable to process electrical signals from the pair of electrodes and determine a value descriptive of ventricular fibrillation. The controller is further operable to compare the value, and in accordance to the comparison of the value to the threshold, generate interface control signals to control the user interface to communicate instructions to a rescuer to perform cardio-pulmonary resuscitation with or without ventilation.

In the drawings:

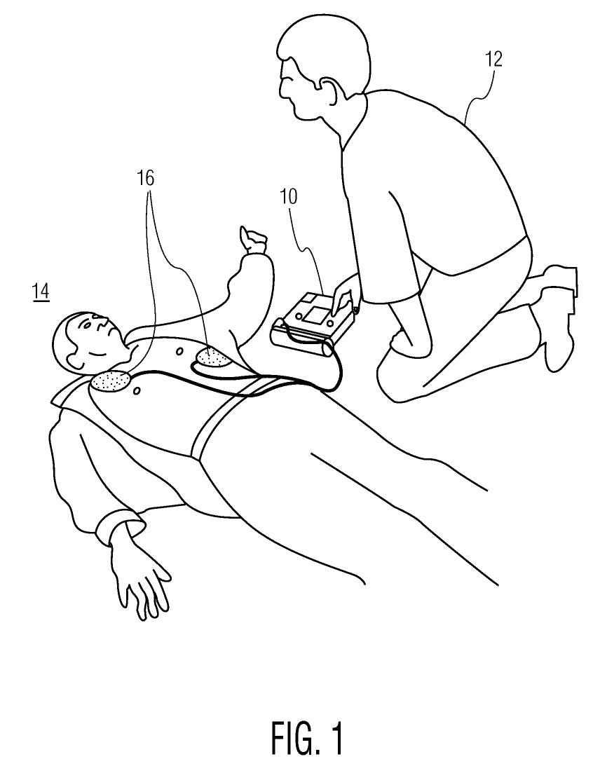

FIG. 1 is an illustration of a defibrillator being applied to a patient suffering from cardiac arrest.

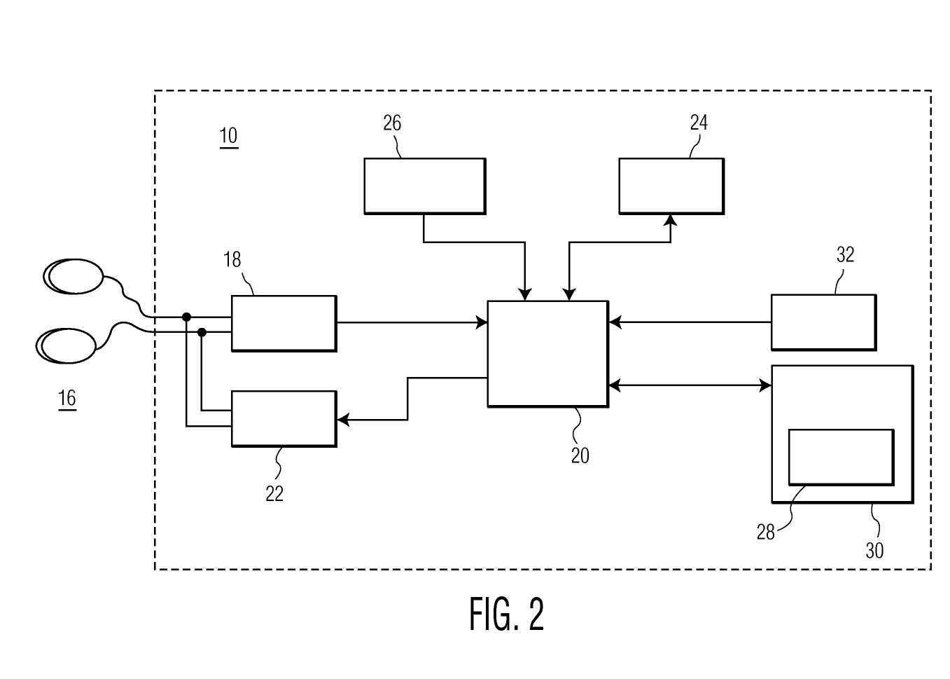

FIG. 2 is a block diagram of a defibrillator constructed in accordance with an embodiment of the present invention.

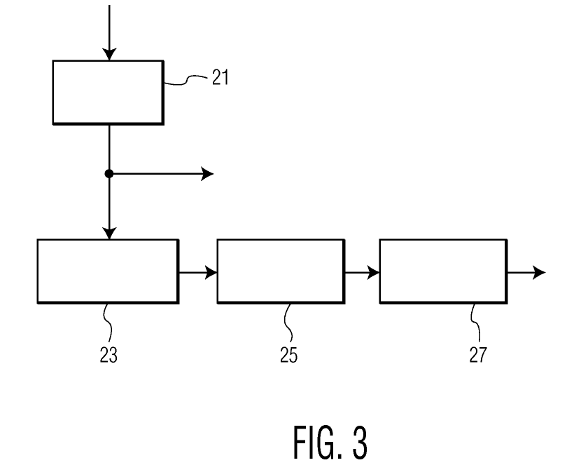

FIG. 3 is a block diagram of a portion of an ECG front end circuit and controller according to an embodiment of the present invention of the defibrillator of FIG. 2.

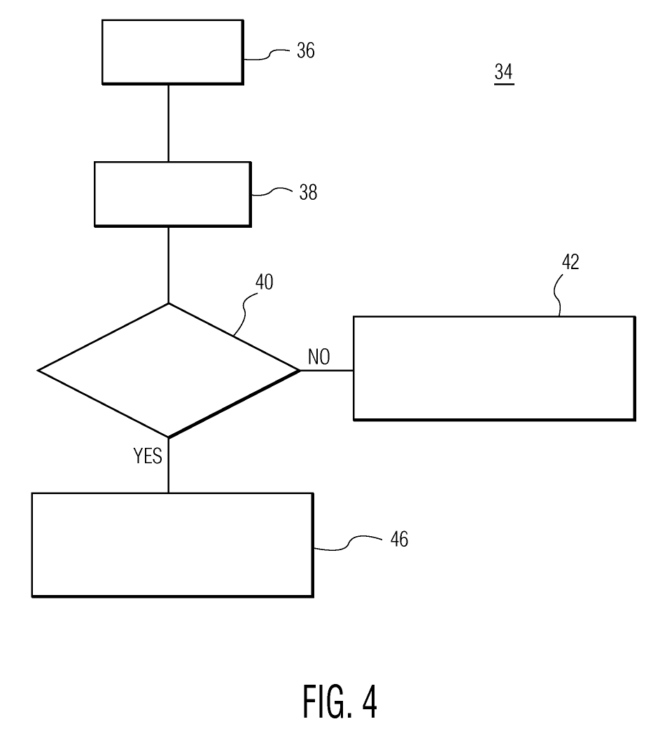

FIG. 4 is a process flow diagram of a method for treating VF in accordance with an embodiment of the present invention.

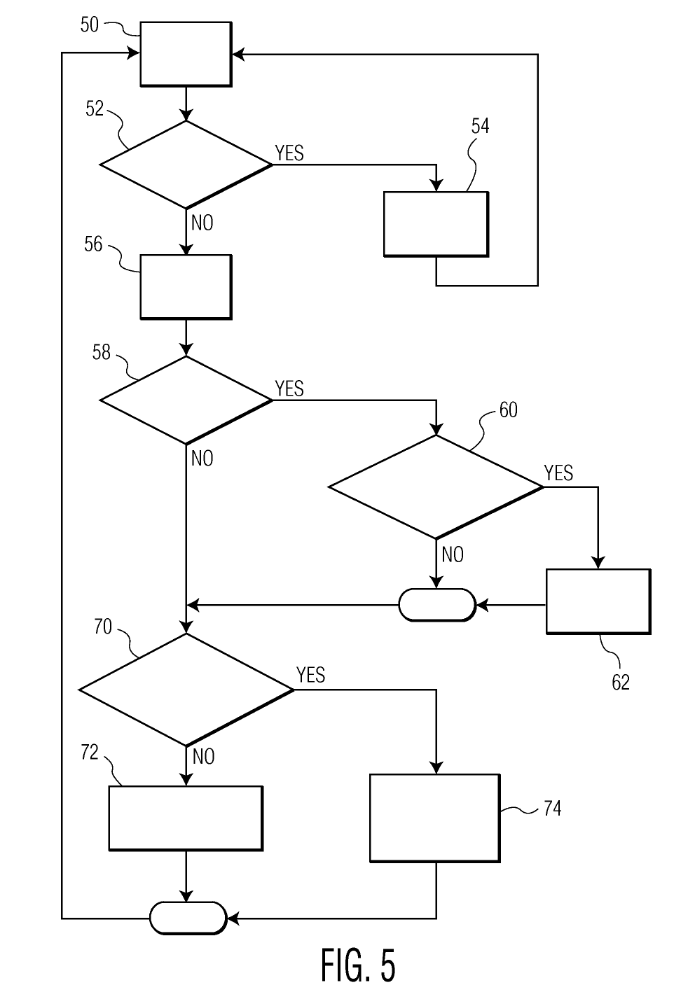

FIG. 5 is a process flow diagram of an alternative method for treating VF in accordance with an embodiment of the present invention.

FIG. 1 is an illustration of a defibrillator 10 being applied by a user 12 to resuscitate a patient 14 suffering from cardiac arrest. Defibrillators deliver a high-voltage impulse to the heart in order to restore normal rhythm and contractile function in patients who are experiencing arrhythmia, such as VF or VT that is not accompanied by spontaneous circulation. There are several classes of defibrillators, including manual defibrillators, implantable defibrillators, and automatic external defibrillators (AEDs). AEDs differ from manual defibrillators in that AEDs can automatically analyze the electrocardiogram (ECG) rhythm to determine if defibrillation is necessary. In nearly all AED designs, the user is prompted to press a shock button to deliver the defibrillation shock to the patient when a shock is advised by the AED. The defibrillator 10 may be in the form of an AED capable of being used by a lay first responder. The defibrillator 10 may also be in the form of a manual defibrillator for use by paramedics or other highly trained medical personnel.

A pair of electrodes 16 are applied across the chest of the patient 14 by the user 12 in order to acquire an ECG signal from the patient's heart. The defibrillator 10 may be programmed to analyze the ECG signal for signs of arrhythmia such as VF or VT.

FIG. 2 illustrates an embodiment of a defibrillator 10 constructed in accordance with the principles of the present invention. For purposes of the discussion that follows, the defibrillator 10 is configured as an AED, and is designed for small physical size, light weight, and relatively simple user interface capable of being operated by personnel without high training levels or who otherwise would use the defibrillator 10 only infrequently. In contrast, a paramedic or clinical defibrillator of the type generally carried by an emergency medical service (EMS) responder tends to be larger, heavier, and have a more complex user interface capable of supporting a larger number of manual monitoring and analysis functions. Although the present embodiment of the invention is described with respect to application in an AED, other embodiments include application in different types of defibrillators, for example, manual defibrillators, and paramedic or clinical defibrillators.

An ECG front end circuit 18 is connected to the pair of electrodes 16 that are connected across the chest of the patient 14. The ECG front end circuit 18 operates to amplify, buffer, filter and digitize an electrical ECG signal generated by the patient's heart to produce a stream of digitized ECG samples. The digitized ECG samples are provided to a controller 20 that performs an analysis to detect VF, shockable VT or other shockable rhythm and, in accordance with the present invention, that performs an analysis to determine a treatment regimen which is likely to be successful. If a shockable rhythm is detected in combination with determination of a treatment regimen that indicates immediate defibrillation shock, the controller 20 sends a signal to HV (high voltage) delivery circuit 22 to charge in preparation for delivering a shock and a shock button on a user interface 24 is activated to begin flashing. When the user presses the shock button on the user interface 24 a defibrillation shock is delivered from the HV delivery circuit 22 to the patient 14 through the electrodes 116.

The controller 20 may be coupled to further receive input from a microphone 26 to produce a voice strip. The analog audio signal from the microphone 26 is preferably digitized to produce a stream of digitized audio samples which may be stored, along with ECG and AED event markers, as part of an event summary 28 in a memory 30. The user interface 24 may consist of a display, an audio speaker, and control buttons such as an on-off button and a shock button for providing user control as well as visual and audible prompts. The user interface 24 may serve to display and provide instructions in order to instruct a rescuer how to properly provide treatment. A clock 32 provides real-time clock data to the controller 20 for time-stamping information contained in the event summary 28. The memory 30, implemented either as on-board RAM, a removable memory card, or a combination of different memory technologies, operates to store the event summary 28 digitally as it is compiled over the treatment of the patient 14. The event summary 28 may include the streams of digitized ECG, audio samples, and other event data as previously described.

As known, VF changes with time from its onset. In particular, as time passes from the onset of VF, heart muscle fluctuations become less vigorous. Physiological experiments also support a premise that ventilation may not be necessary early in VF, primarily because of remnant oxygen in the blood. In the early stages of VF, it may be therefore be better for a rescuer to focus on performing chest compressions without ventilation in order to circulate remnant oxygen in the blood before it has been depleted. As a result, the rescuer can be relieved of the burden (and reluctance) to provide ventilation at this time and concentrate on performing chest compressions.

According to embodiments of the present invention, characteristics of VF are measured to assess how long VF has been occurring in order to determine whether ventilation should be performed with chest compressions during administration of CPR and instruct a rescuer accordingly.

One example method for characterizing VF based on an ECG is described in International Publication Number WO 2006/136974, entitled “Defibrillator With Automatic Shock First/CPR First Algorithm” [hereinafter the '974 publication] which is incorporated herein by reference. As described in the '974 publication, a return of spontaneous circulation (ROSC) score can be calculated from ECG data collected by a defibrillator, such as defibrillator 10 (FIG. 2). The ROSC score can then be compared to a threshold or thresholds to advise the rescuer on treatment of the patient. One example described in the '974 publication uses the ROSC score to determine whether to advise a rescuer to administer a shock first or perform CPR.

As discussed in the '974 publication, a ROSC score can be calculated in several ways. For one example, the ROSC score can be calculated as the mean magnitude of the bandwidth limited first derivative (or first difference, which is a discrete-time analog) of the ECG over a period of a few seconds. Another example is to calculate a ROSC score from the median magnitude of the first derivative of the ECG. The '974 publication provides a more detailed description of the calculation of a ROSC score.

In an embodiment of the present invention, a ROSC score is used to determine whether ventilation should be performed with chest compressions during administration of CPR and instructs a rescuer accordingly. The thresholds for the values of the ROSC used to determine when to administer a shock first or perform CPR first in the '974 publication may also be used in the present invention for evaluating when to include ventilation when administering CPR. In an alternative embodiment, a different threshold may also be used. In other embodiments, characteristics of the ECG such as amplitude, frequency content, frequency content in a specific frequency band, or any combination of these characteristics, may be used to characterize a patient's VF, and consequently, provide a basis for determining whether ventilation should be performed with chest compressions during administration of CPR and instruct a rescuer accordingly.

FIG. 3 illustrates a portion of the ECG front end circuit 18 and controller 20 which operate according to an embodiment of the present invention. As previously mentioned the electrodes 116 provide ECG signals from the patient which are sampled (digitized) by an A/D converter 21. The digitized ECG signals are coupled to a processor in the controller 20 which analyzes the ECG waveform to determine whether application of a shock is advised. The ECG samples are coupled to a downsampler 23 which subsamples the stream of ECG samples to a lower data rate. For instance, a data stream of 200 samples/sec may be downsampled to 100 samples/sec. The downsampled ECG data is coupled to a ROSC calculator 25 which determines a ROSC score from the ECG data. The ROSC score is compared against a threshold by threshold comparator 27 to determine a mode of treatment which is most likely to lead to a successful resuscitation. As previously discussed, the ROSC score is compared to a threshold to determine whether a rescuer should be instructed to perform chest compressions with or without ventilation during administration of CPR.

FIG. 4 illustrates a process 34 for treating VF according to an embodiment of the present invention. The controller 20 (FIG. 2) may be programmed to perform the process 34. Process 34 includes measuring electrical activity of a patient's heart and generating ECG data representing the patient ECG at block 36. A VF characteristic (or characteristics) is determined at step 38 from the ECG data. As previously discussed, the characteristic may include characteristics of the ECG such as amplitude, frequency content, frequency content in a specific frequency band, or any combination of these characteristics. In some embodiments, the VF characteristic is a ROSC score as described in the '974 publication.

At step 40 the VF characteristic is compared to a ventilation threshold. If the VF characteristic is not below the threshold, suggesting that VF recently began and remnant oxygen present in the blood can be circulated, the defibrillator 10 instructs a rescuer to perform chest compressions without ventilation during administration of CPR, if necessary, at step 42. Such instructions allow the rescuer to focus on providing chest compressions, which has the benefits of addressing any reluctance by the rescuer to perform CPR because of ventilating the patient, as well as limiting interruptions during chest compressions. In contrast, if the VF characteristic is below the threshold, suggesting that VF has been ongoing and remnant oxygen has been depleted, the defibrillator 10 instructs the rescuer to perform chest compressions with ventilation during administration of CPR, if necessary, at step 46.

FIG. 5 illustrates an example of a protocol of the present invention that combines CPR-first or shock-first therapy determination (such as that described in the '974 publication) with CPR, including a determination based on a VF characteristic of whether the CPR should include ventilation. Referring to FIG. 5, the protocol begins with an initial assessment 50 of the patient as being conscious or unconscious. If the assessment 52 is that the patient is conscious, the rescuer attends to the patient's condition at 54, as a conscious patient has not experienced VF. If the patient is unconscious, ECG data is acquired from the patient to measure the electrical activity of the patient's heart at 56. The ECG data is processed and, if a shockable rhythm is determined at 58, a VF characteristic referred to as f1 is compared with a shock threshold at 60. The VF characteristic may include characteristics of the ECG such as amplitude frequency content, frequency content in a specific frequency band, or any combination of these characteristics. In some embodiments, the VF characteristic is the ROSC score as described in the '974 publication. If the VF characteristic is above the applicable shock threshold, a shock is advised at 62 and the rescuer is prompted to press the shock button as previously described.

However, if no shockable rhythm is identified at 58 or no shock is advised at 62, then the same or another VF characteristic referred to as f2 is compared with a ventilation threshold at 70. If this VF characteristic is above the applicable ventilation threshold, indicating that some residual oxygen remains in the blood stream, then a prompt is produced advising that CPR be performed without the inclusion of ventilation, e.g., only chest compressions. However if the comparison of the VF characteristic with the ventilation threshold shows that the characteristic is below the ventilation threshold, then CPR with ventilation is advised to introduce oxygen into the blood stream through ventilation such as mouth-to-mouth resuscitation, and to force the oxygenated blood through the body with chest compressions. Preferably the CPR is performed with the aid of a compression puck attached to the defibrillator by which the defibrillator prompts can assist the delivery of CPR if needed, as described in U.S. Pat. No. 6,306,107 (Myklebust et al. The Myklebust et al. system receives a signal in response to each chest compression and is therefore capable of monitoring when the CPR chest compressions begin and end. At the end of CPR the protocol of FIG. 5 repeats again with patient assessment 50.

During the process 34 of FIG. 4 and the protocol of FIG. 5, if at any time return of spontaneous circulation occurs, then the processes stop and proceed to the next stage of treatment.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for treating a heart disorder comprising:
measuring electrical activity of a patient's heart;
processing the measured electrical activity to determine a value descriptive of ventricular fibrillation;
comparing the value to a threshold;
producing an output according to the comparison of the value to the threshold, the output instructing a rescuer to perform cardio-pulmonary resuscitation with or without ventilation.

2. The method of claim 1, wherein measuring electrical activity comprises acquiring electrocardiographic data.

3. The method of claim 2, wherein the value corresponds to an amplitude of the electrocardiographic data.

4. The method of claim 2, wherein the value corresponds to a frequency content of the electrocardiographic data.

5. The method of claim 4, wherein the value is a function of both the frequency content and an amplitude of the electrocardiographic data.

6. The method of claim 2, wherein measuring electrical activity is performed using a defibrillator.

7. The method of claim 6, wherein the threshold is a first threshold and the output is a first output, the method further comprising:
comparing the value to a second threshold; and
according to the comparison of the value and the second threshold producing a second output instructing an operator to administer a shock and then to perform cardiopulmonary resuscitation (CPR) or to perform CPR and then to administer a shock.

8. The method of claim 6, wherein the second output is produced before the first output.

9. The method of claim 1 wherein measuring electrical activity comprises acquiring electrocardiographic data and wherein processing the measured electrical activity to determine a value descriptive of ventricular fibrillation comprises processing the electrocardiographic data to determine a value related to the first derivative of the electrocardiographic data.

10. A defibrillator comprising
a pair of electrodes configured to detect electrical signals from a patient;
a user interface configured to provide instructions to a rescuer in accordance with interface control signals;
a controller electrically coupled to the electrodes and user interface, the controller operable to process electrical signals from the pair of electrodes and determine a value descriptive of ventricular fibrillation, the controller further operable to compare the value, and in accordance to the comparison of the value to the threshold, generate interface control signals to control the user interface to communicate instructions to a rescuer to perform cardio-pulmonary resuscitation with or without ventilation.

11. The defibrillator of claim 10, wherein the controller is operable to acquire electrocardiographic data.

12. The defibrillator of claim 11, wherein the controller is operable to determine the value from an amplitude of the electrocardiographic data.

13. The defibrillator of claim 11, wherein the value corresponds to a frequency content of the electrocardiographic data.

14. The defibrillator of claim 13, wherein the value is a function of both the frequency content and an amplitude of the electrocardiographic data.

15. The defibrillator of claim 10, wherein the threshold is a first threshold and wherein the controller is further operable to compare the value to a second threshold, and according to the comparison of the value and the second threshold, generate interface control signals to control the user interface to communicate instructions to the rescuer indicating a sequence for administering a shock and cardio-pulmonary resuscitation.

16. The defibrillator of claim 15, wherein the controller is operable to first compare the second threshold to the value and then to compare the first threshold to the value.

17. The defibrillator of claim 10, wherein the user interface is a speaker and wherein the instructions communicated to the rescuer correspond to a plurality of audible messages.

18. The defibrillator of claim 10, wherein the user interface is a display and wherein the instructions communicated to the rescuer correspond to a plurality of messages displayed on the display.
